(19) United States
(12) Patent Application Publication
(10) Pub. No.: US 2002/0032478 A1
(43) Pub. Date: Mar. 14, 2002

(54) Myocardial stents and related methods of providing direct blood flow from a heart chamber to a coronary vessel

(75) Inventors: Peter Boekstegers, Diessend A (DE); Nancy Briefs, Nashua, NH (US); Jerrick Buck, Nashua, NH (US); Laurence A. Roth, Windham, NH (US); Robert Swain, Bedford, NH (US)

Correspondence Address: Finnegan, Henderson, Farabow,, Garrett & Dunner, L.L.P., 1300 I Street, N.W., Washington, DC, 20005-3315, US

(73) Assignee: Percardia, Inc.

(21) Appl. No.: 09/917,655

(22) Filed: Jul. 31, 2001

Related U.S. Application Data
(60) Provisional application No. 60/223,424, filed on Aug. 7, 2000.

(51) Int. Cl. A61F 2/06

(52) U.S. Cl. 623/1.16

(57) ABSTRACT

The method and apparatus described and illustrated herein generally relate to a bypass method to provide blood flow directly from a heart chamber, such as the left ventricle, and coronary vasculature, such as a coronary artery, and a conduit especially suited for placement in the myocardium to provide such flow. The conduit is particularly useful when a blockage partially or completely obstructs the coronary artery, in which case the conduit is positioned distal to the blockage. Aspects of the present invention relate to conduits in the form of stents that have particular configurations exhibiting properties suited to placement in the myocardium.

Such a stent expands from a first diameter during delivery to a myocardial site to a second diameter when implanted in the site. The stent includes a configuration that has high radial strength to resist deformation from contractile forces experienced during a cardiac cycle. The configuration also exhibits high flexibility in a compressed state and a deployed state to permit passage to a myocardial site and remain patent when implanted in the site. The expandable stent may include suitable coverings and coatings.

### REFERENCE TO RELATED APPLICATION

This application claims the benefits of priority, provided under 35 U.S.C. §119, of U.S. Provisional Patent Application No. 60/223,424, filed Aug. 7, 2000 and entitled “Myocardial Stents and Related Methods of Providing Direct Blood Flow from a Heart Chamber to a Coronary Vessel,” the complete disclosure of which is incorporated by reference herein.

### FIELD OF THE INVENTION

The present invention relates to conduits for placement in the myocardium between a heart chamber and coronary vasculature, and related methods of using such a conduit to provide direct blood flow from the heart chamber to a coronary vessel, and more particularly, to such methods employing conduits in the form of stents having particular configurations that exhibit properties suited to placement in the myocardium.

### BACKGROUND OF THE INVENTION

Coronary artery disease is a major problem in the U.S. and throughout the world. Coronary arteries as well as other blood vessels frequently become clogged with plaque which, at the very least, can reduce blood and oxygen flow to the heart muscle (myocardium), and may impair the efficiency of the heart's pumping action, and can lead to heart attack (myocardial infarction) and death. In some cases, these coronary arteries can be unblocked through noninvasive techniques such as balloon angioplasty. In more difficult cases, a surgical bypass of the blocked vessel is necessary.

In a coronary bypass operation, one or more venous segments are inserted between the aorta and the coronary artery, or, alternatively, the distal end of an internal mammary artery is anastomosed to the coronary artery at a site distal to the stenosis or occlusion. The inserted venous segments or transplants act as a bypass of the blocked portion of the coronary artery and thus provide for a free or unobstructed flow of blood to the heart. More than 500,000 bypass procedures are performed in the U.S. every year.

Such coronary artery bypass graft (CABG) surgery, however, is a very intrusive procedure which is expensive, time-consuming, and traumatic to the patient. The operation requires an incision through the patient's sternum (sternotomy), and that the patient be placed on a heart-lung bypass pump so that the heart can be operated on while not beating. A saphenous vein graft is harvested from the patient's leg, another highly invasive procedure, and a delicate surgical procedure is required to piece the bypass graft to the coronary artery (anastomosis). Hospital stays subsequent to the surgery and convalescence are prolonged. Furthermore, many patients are poor surgical candidates due to other concomitant illnesses.

As mentioned above, another conventional treatment is percutaneous transluminal coronary angioplasty (PTCA) or other types of angioplasty. However, such vascular treatments are not always indicated due to the type or location of the blockage or stenosis, or due to the risk of emboli.

Thus, there is a need for an improved coronary bypass system which is less traumatic to the patient.

### SUMMARY OF THE INVENTION

The bypass method and apparatus described and illustrated herein generally relates to a conduit placed in the myocardium between a heart chamber and coronary vasculature to bypass a blocked or stenosed blood vessel segment. The conduit may be placed between the left ventricle and a coronary artery, oftentimes the left anterior descending artery (LAD), to provide blood flow directly therethrough. The conduit is particularly useful when a blockage partially or completely obstructs the coronary artery, in which case the conduit is positioned distal to the blockage.

More particularly, an aspect of the present invention relates to bypass methods using conduits in the form of stents that have particular configurations exhibiting properties suited to placement in the myocardium. Such a stent expands from a first diameter during delivery to a myocardial site to a second diameter when implanted in the site. The stent includes a configuration that has high radial strength to resist deformation from contractile forces experienced during a cardiac cycle. The configuration also exhibits high flexibility in a compressed state and a deployed state to permit passage to a myocardial site and remain patent when implanted in the site. According to aspects of the inventions, the expandable stent may include suitable coverings and coatings applied to the stent, and may also be modified to improve seating in the floor of the artery by, for example, an end having a flared configuration.

The foregoing general description and the following detailed description are exemplary and explanatory only and are not restrictive of the invention, as claimed.

### BRIEF DESCRIPTION OF THE DRAWINGS

The accompanying drawings, which are incorporated in and constitute a part of this specification, illustrate embodiments of the invention and together with the description, serve to explain the principles of the invention.

FIG. 1 is a schematic, cross-sectional view of a human heart, showing a conduit in the myocardium of the heart between the left ventricle and a coronary artery.

FIG. 2 is a plan view of a stent suitable for delivery to and implantation in the heart wall as a left ventricular conduit, according to an embodiment of the present invention.

FIG. 3 is a plan view of another stent suitable for delivery to and implantation in the heart wall as a left ventricular conduit, according to an embodiment of the present invention.

FIG. 4 is a plan view of a configuration for a further stent suitable for delivery to and implantation in the heart wall as a left ventricular conduit, according to an embodiment of the present invention.

FIG. 5 is a plan view of a covered stent having a flared end for seating in the floor of a coronary artery, according to an embodiment of the present invention.

### DETAILED DESCRIPTION OF THE PREFERRED EMBODIMENT

As is well known, coronary arteries branch off the aorta and are positioned along the external surface of the heart wall. Oxygenated blood that has returned from the lungs to the heart then flows from the heart to the aorta. Some blood in the aorta flows into the coronary arteries, and the remainder of blood in the aorta flows on to the rest of the body. The coronary arteries are the primary blood supply to the heart muscle and are thus critical to life. In some individuals, atherosclerotic plaque, aggregated platelets, and/or thrombi build up within the coronary arteries, blocking the free flow of blood and causing complications ranging from mild angina to heart attack and death. The presence of coronary vasospasm, also known as “variant angina” or “Prinzmetal's angina,” compounds this problem in many patients.

The principles of the present invention are not limited to left ventricular conduits, and extend to conduits between any heart chamber and coronary vasculature, including coronary arteries and veins. Furthermore, fluid flow through the conduit is not limited to any particular direction of flow and can be antegrade or retrograde with respect to the normal flow of fluid. In addition, the conduit can traverse various intermediate destinations and is not limited to any particular flow sequence. For example, the conduit can communicate from the left ventricle, through the myocardium, into the pericardial space, and then into the coronary artery. The presently preferred embodiment, however, includes direct transmyocardial communication from a left ventricle, through the myocardium, and into the coronary artery.

The bypass which is achieved with conduits according to the present invention is not limited to a complete bypass of blood flow, but can also include a partial bypass which advantageously supplements the normal blood flow. Moreover, the occlusions which are bypassed may be of a partial or complete nature, and therefore the terminology “bypass” or “occlusion” should not be construed to be limited to a complete bypass or a complete occlusion but can include partial bypass and partial occlusion as described.

The conduits disclosed herein can also provide complete passages or partial passages through the myocardium. The presently preferred application, however, is a complete passage through the myocardium.

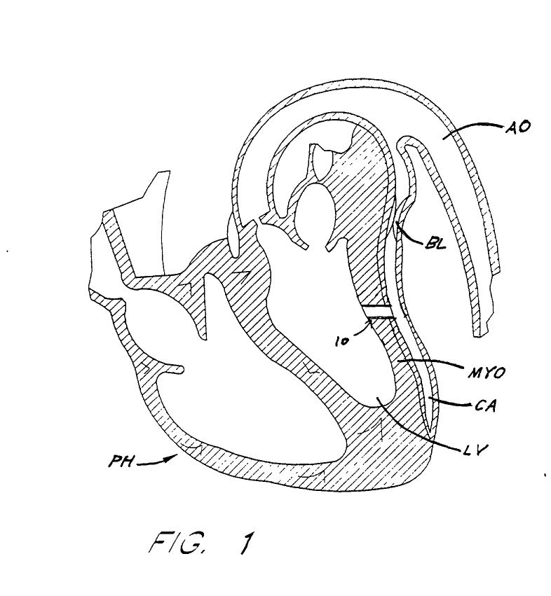

As illustrated in FIG. 1, a coronary artery bypass is accomplished by disposing a left ventricular conduit 10 in a heart wall or myocardium MYO of a patient's heart PH. The conduit 10 preferably extends from the left ventricle LV of heart PH to a clogged coronary artery CA at a point downstream of a blockage BL.

In the preferred embodiments of this invention, conduit 10 is an expandable stent that has a configuration that exhibits properties especially suitable for placement in the myocardium. More particularly, the stent has relatively high radial and compressive strength. Such sufficient strength is particularly important for a stent placed in the myocardium due to the relatively high contractile forces experienced during the cardiac cycle.

Expandable stent 10 also preferably has a configuration that exhibits relatively high flexibility in a compressed state as well as a deployed state. Sufficient flexibility permits percutaneous delivery along a tortuous path to the myocardial site and also permits the stent to remain patent when bent and placed at an angle in the myocardium. A stent configuration that exhibits high flexibility also allows the stent to conform to the shape of the myocardial passage.

The expandable stent preferably is tubular, having a first diameter permitting delivery to a myocardial site and a second expanded diameter when placed within the myocardium. The stent achieves this second, variable diameter through the application of a radially outward force applied to the interior of the stent. The amount of force controls the extent of the expansion of the stent and thus its second diameter. The stent may be placed in the myocardium through any of a number of suitable methods, as will be described herein.

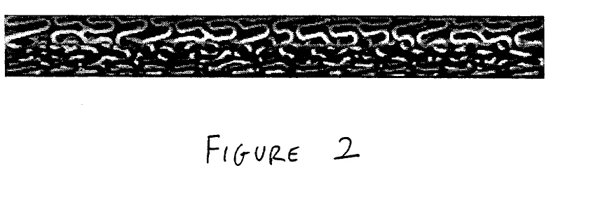

A stent that has been found to be particularly suitable for delivery to and implantation in the heart wall as a left ventricular conduit, and exhibits the various properties just mentioned, is a commercially available stent sold by Orbus Medical Technologies, Inc. of Fort Lauderdale, Fla. under the trade name “R stent.” The “R stent” has a configuration made of high grade 316 stainless steel cut into the shape of an “R” and formed into a tubular stent, as shown in FIG. 2. The commercial “R stent” has characteristics and a configuration very much like the stents described in European Patent Application No. 98201446.6 published on Dec. 16, 1998 as Publication No. EP 0 884 029 A1, the complete disclosure of which is incorporated by reference herein, and European Patent Application No. 97201799.0 published on Jan. 13, 1999 as Publication No. EP 0 890 346 A1, the complete disclosure of which also is incorporated by reference herein. As explained in those European applications, the stent configuration is a substantially continuous structure of mutually staggered undulations having a pattern that advances helically along the stent.

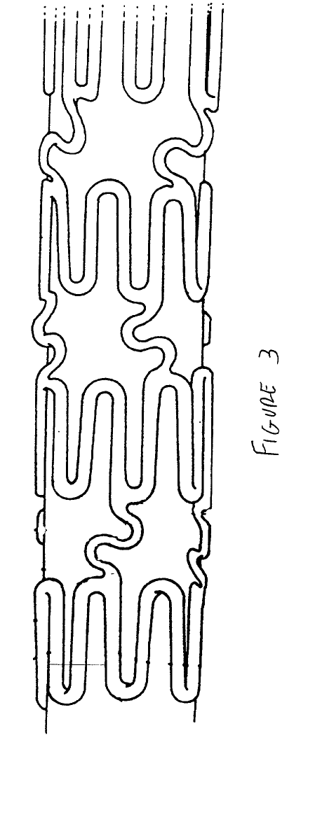

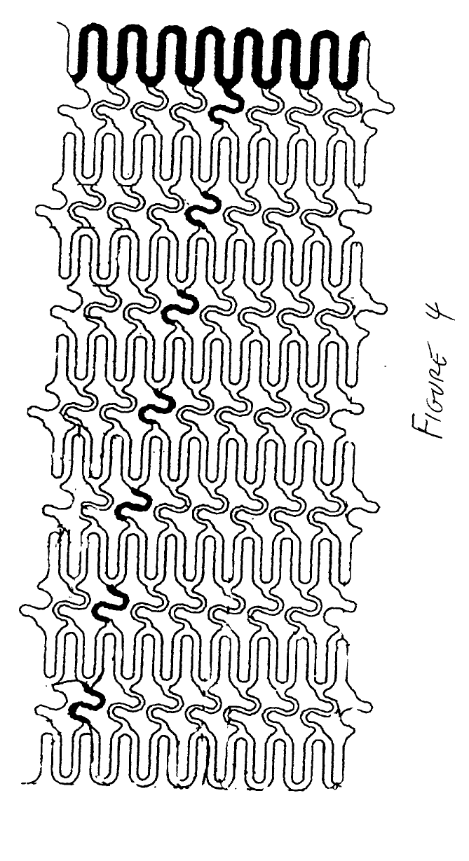

Another stent that has been found to be particularly suitable for delivery to and implantation in the heart wall as a left ventricular conduit, and exhibits the properties mentioned above, is a commercially available stent sold by Stent Tech of France. The Stent Tech stent has a configuration made of high grade stainless steel cut into a series of annular segments and connectors, like the stents depicted in FIGS. 3 and 4 and more completely described in European Patent Application No. 98401015.7 published on Nov. 11, 1998 as Publication No. EP 0 876 806 A1, the complete disclosure of which is incorporated by reference herein, and in European Patent Application No. 99403076.5 published on Jun. 14, 2000 as Publication No. EP 1 008 329 A1, the complete disclosure of which also is incorporated by reference herein. The annular segments have a wavy shape, with at least some of the loops of the waves attached to the S-shaped connectors. The connectors lend a high degree of transverse flexibility to the stent.

In preferred embodiments of the invention, the expandable stents from Orbus Medical Technologies and Stent Tech have a covering of expandable PTFE material. In the preferred embodiment of the invention, the metal stent is sandwiched between the PTFE material, i.e. the PTFE covers the entire stent, including the inside and outside surfaces.

A still further stent that has been found to be particularly suitable for delivery to and implantation in the heart wall as a left ventricular conduit, and exhibits the properties mentioned above, is a commercially available stent manufactured and sold by Jomed International AB and Jomed Implantate GmbH of Germany under the trade name “JOSTENT Coronary Stent Graft.” The “JOSTENT Coronary Stent Graft” is made of two layers of high grade 316 stainless steel struts with expandable PTFE material sandwiched between the layers. The stent is available in a variety of lengths.

In a further preferred embodiment, the covered expandable stent includes a coating on the inner surface that is in contact with blood flow. The coating preferably comprises a commercially available material sold by Carmeda North America of San Antonio, Tex. and Carmeda AB of Stockholm, Sweden under the trade name “Carmeda BioActive Surface (CBAS).” CBAS is a heparin-based coating that provides a hemocompatible, antithrombogenic surface to withstand aggressive blood flow and stent flexure. The CBAS coated inner surface reduces thrombus formation and platelet adhesion. In the coating process, heparin is covalently bound to the stent inner surface through a suitable method, for example using aqueous solutions circulated through the fluid path of the stent. Other suitable coating methods are described in, for example, U.S. Pat. Nos. 4,613,665 and 5,049,403, the complete disclosures of both of which are incorporated by reference herein.

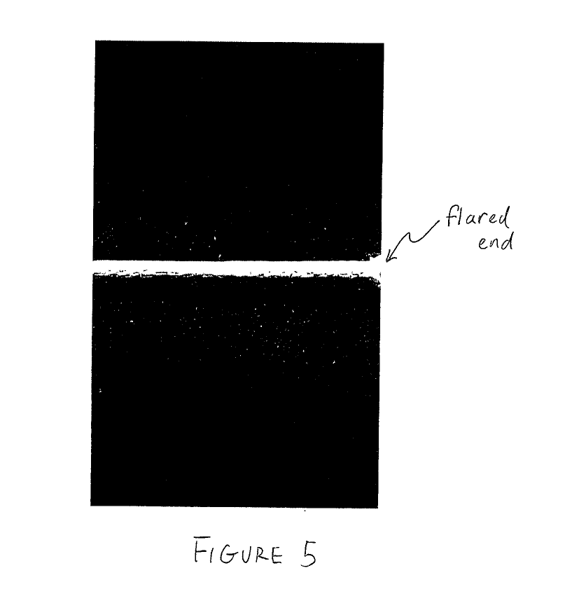

In an even further preferred embodiment according to the present invention, the stent incorporates at least one end that is flared outwardly. At least the end intended to be placed toward the coronary vasculature preferably includes such a flared configuration to seat in the coronary vein or artery and aid in anchoring the stent in the myocardial passage and prevent migration. As an example, FIG. 5 shows the Orbus Medical Technologies “R-stent” with such a flared end.

The expandable stents may be implanted into the myocardium between the left ventricle and a coronary artery in a variety of methods consistent with sound medical practice, including vascular or surgical deliveries, and minimally invasive techniques. For example, various delivery rods, including solid trocar-like rods, and associated methods may be used. As a further example, the stent may be implanted through any of the delivery techniques described in U.S. Provisional Patent Application Ser. No. 60/201,732 entitled “A METHOD OF DELIVERING A VENTRICULAR STENT” and filed on May 4, 2000, the complete disclosure of which is incorporated by reference herein. That provisional application and the present application are commonly assigned.

A presently preferred technique described in that provisional application that is suitable for the preferred stent configurations described above includes a direct surgical approach using balloon deployment. That approach first may involve performing a left thoracotomy or stenotomy. An arteriotomy or direct puncture is then performed to obtain access to the artery, for example the left anterior descending artery (LAD). A needle is placed through the artery into the left ventricle. Flow may be confirmed through the needle. A guide wire then is inserted through the needle and the needle is removed. A stent having a preferred configuration according to the present invention may be pre-flared, as shown in FIG. 5, and mounted on the proximal balloon of a double balloon catheter. The catheter then is placed over the guide wire and the myocardial channel is dilated using the distal balloon of the catheter. The distal balloon then is deflated and the proximal balloon is positioned in the predilated channel and inflated to deploy the stent. Once the stent is seated properly, the catheter may be removed. A patch may be sewn over the arteriotomy for closure, or the site is closed using conventional suture techniques.

The direct surgical approach just described is an example of a technique used to implant a stent according to the present invention. Other suitable techniques include any method of percutaneous delivery of the stent.

Experiments have been performed using the Orbus Medical Technologies “R stent” with an expandable PTFE covering, and with and without antithrombogenic coating. In these experiments, the stent was balloon deployed in the myocardium of a living pig using the direct surgical approach discussed above. The procedure was performed on a beating heart without the use of cardiopulmonary bypass. The stent was deployed using 2.5 mm and 3.0 mm balloons. The implanted stent spanned the myocardium between the left ventricle and the left anterior descending artery and seated at the floor of that artery. The stent provided flow communication between the left ventricle and the coronary artery and resisted deformation or collapse from the contractile forces of the myocardium.

Experimental tests also have been performed with a Jomed “JOSTENT Coronary Stent Graft” that included a PTFE covering, an antithrombogenic coating, and a pre-flared end. Once again, the stent was balloon deployed in the myocardium of a living pig using the direct surgical approach. The stent was 26 mm long and had a collapsed diameter of 1.5 mm and a deployed diameter of 2.5 mm. The test results showed that the stent remained evenly open and provided adequate flow from the left ventricle to the LAD.

The embodiments illustrated and described above are provided merely as examples of certain preferred embodiments of the present invention. Various changes and modifications can be made from the embodiments presented herein by those skilled in the art without departure from the spirit and scope of the invention, as described by the appended claims.

What is claimed is:

1. A method of providing blood flow directly from a heart chamber to a coronary vessel, comprising:
providing a stent that includes a configuration having sufficient radial strength to resist deformation from contractile forces experienced during a cardiac cycle and sufficient flexibility in a compressed state and a deployed state to permit passage to a myocardial site and remain patent when implanted in the site;
delivering the stent in the compressed state into a passage at the myocardial site; and
expanding the stent to deploy the stent in the passage.

2. The method of claim 1, wherein the stent includes a covering.

3. The method of claim 2, wherein the covering includes expandable PTFE.

4. The method of claim 2, wherein the covering covers substantially all of an inside surface and an outside surface of the stent.

5. The method of claim 2, wherein the stent includes a coating over the covering on an inside surface of the stent.

6. The method of claim 5, wherein the coating includes heparin.

7. The method of claim 5, wherein the coating is hemocompatible and antithrombogenic.

8. The method of claim 1, wherein the stent includes a covering having expandable PTFE that covers substantially all of an inside surface and an outside surface of the stent, and the stent includes a heparin-based coating over the covering on the inside surface of the stent.

9. The method of claim 1, wherein the stent includes a flared end.

10. The method of claim 9, wherein the flared end is placed in the passage to face the coronary vessel.

11. The method of claim 1, wherein the coronary vessel is a coronary artery.

12. The method of claim 1, wherein the heart chamber is a left ventricle.

13. The method of claim 1, wherein the myocardial site is distal to a coronary blockage.

14. The method of claim 13, wherein the coronary blockage is a partial blockage.

15. The method of claim 1, wherein delivering the stent includes delivering the stent percutaneously.

16. A method of providing blood flow directly from a left ventricle to a coronary artery, comprising:
providing a stent that includes a configuration having sufficient radial strength to resist deformation from contractile forces experienced during a cardiac cycle and sufficient flexibility in a compressed state and a deployed state to permit passage to a myocardial site distal to a coronary blockage and remain patent when implanted in the site, wherein the stent includes a covering having expandable PTFE that covers substantially all of an inside surface and an outside surface of the stent, and the stent includes an antithrombogenic coating over the covering on the inside surface of the stent;
delivering the stent percutaneously in the compressed state into a passage at the myocardial site; and
expanding the stent to deploy the stent in the passage.

17. A method of providing blood flow directly from a heart chamber to a coronary vessel, comprising:
providing a stent that includes a configuration having sufficient radial strength to resist deformation from contractile forces experienced during a cardiac cycle and sufficient flexibility in a deployed state to permit passage to a myocardial site and remain patent when implanted in the site;
applying a covering to the stent;
applying a coating over the covering on an inside surface of the stent; and
delivering the stent into a passage at the myocardial site.

18. The method of claim 17, wherein delivering the stent includes percutaneously delivering the stent in a compressed state and expanding the stent to deploy the stent in the passage.

19. The method of claim 17, wherein the covering includes expandable PTFE.

20. The method of claim 17, wherein the covering covers substantially all of the inside surface and an outside surface of the stent.

21. The method of claim 17, wherein the coating includes heparin.

22. The method of claim 17, wherein the coating is hemocompatible and antithrombogenic.

23. The method of claim 17, wherein the stent includes a flared end.

24. The method of claim 23, wherein the flared end is placed in the passage to face the coronary vessel.

25. The method of claim 17, wherein the coronary vessel is a coronary artery.

26. The method of claim 17, wherein the heart chamber is a left ventricle.

27. The method of claim 17, wherein the myocardial site is distal to a coronary blockage.

28. The method of claim 27, wherein the coronary blockage is a partial blockage.

29. A conduit for providing blood flow directly from a heart chamber to a coronary vessel, comprising:
a stent that includes a configuration having sufficient radial strength to resist deformation from contractile forces experienced during a cardiac cycle and sufficient flexibility in a compressed state and a deployed state to permit passage to a myocardial site and remain patent when implanted in the site; and
a covering applied to the stent.

30. The conduit of claim 29, wherein the covering includes expandable PTFE.

31. The conduit of claim 29, wherein the covering covers substantially all of an inside surface and an outside surface of the stent.

32. The conduit of claim 29, wherein the stent includes a coating over the covering on an inside surface of the stent.

33. The conduit of claim 32, wherein the coating includes heparin.

34. The conduit of claim 32, wherein the coating is hemocompatible and antithrombogenic.

35. The conduit of claim 29, wherein the covering includes expandable PTFE that covers substantially all of an inside surface and an outside surface of the stent, and the stent includes a heparin-based coating over the covering on an inside surface of the stent.

36. The conduit of claim 29, wherein the stent includes a flared end.
